(19) United States
(12) Patent Application Publication
(10) Pub. No.: US 2020/0390048 A1
(43) Pub. Date: Dec. 17, 2020

(54) CUCURBITURILS AS SEALANTS TO REDUCE POD SHATTERING

(71) Applicant: AQDOT LIMITED, Cambridge, Cambridgeshire (GB)

(72) Inventors: Roger COULSTON, Cambridge (GB); Ben CHEESMAN, Cambridge (GB); Jing ZHANG, Cambridge (GB)

(73) Assignee: AQDOT LIMITED, Cambridge, Cambridgeshire (GB)

(21) Appl. No.: 16/498,945

(22) PCT Filed: Mar. 27, 2018
(86) PCT No.: PCT/GB2018/050791

§ 371 Date: Sep. 27, 2019

(30) Foreign Application Priority Data
Mar. 27, 2017 (GB) 1704862.0

(51) Int. Cl. A01H 3/04; A01N 43/90; A01H 6/20; A01H 6/54; A01N 3/00

(52) U.S. Cl. CPC A01H 3/04; A01N 43/90; A01N 3/00; A01H 6/542; A01H 6/202

(57) ABSTRACT

Use of cucurbituril, a derivative thereof, or a mixture thereof for reducing pod shattering in pod-bearing crops and a pod sealant composition.

The present invention relates to a pod sealant composition comprising: a) cucurbituril, a derivative thereof, or a mixture thereof; and b) a dessicant, and more generally, use of cucurbituril, a derivative thereof, or a mixture thereof for reducing pod shattering in pod-bearing crops.

Pod dehiscence (pod shatter) is a natural seed dispersal process whereby plant species that produce seed containing pods propagate. Pods have hardened shells that protect the seed and in-built weakness lines to allow the seed to be released. For commercial pod-bearing crops such as rapeseed, pod shatter is a major cause of yield loss.

Mature pods of rapeseed and the commercially important cultivar canola are sensitive to opening. Natural shatter occurs where the crop canopy is impacted by heavy rainfall, hail or wind. Physical shatter can occur through swathing (cutting the crop and placing it into tight windrows (a row of cut crop) to reduce weather stimulated pod shatter) or direct heading (cutting and threshing the crop in one operation) during harvest. Typical losses vary between 8-20 percent w/w of the potential yield but reductions of up to 50 percent w/w have been estimated in seasons when weather conditions were poor prior to and during harvest. Therefore reducing the sensitivity of pods to opening increases the proportion of the yield recovered and therefore improves production efficiency.

Rapeseed pod shatter also increases the number of seeds unintentionally dispersed on the land (known as volunteers). Given a typical pod density of 6000-8000 pods per square metre, even a low percentage of yield loss through pod shatter can result in several thousand seeds per square metre being shed onto the soil. Such seeds can remain viable in the soil for several years exacerbating weed control and pest activity. With tighter crop rotations the emergence of volunteer seedlings leads to disadvantages such as oil quality losses and providing green bridges for pests. Reducing pod shatter is important for decreasing volunteer populations.

Strategies to alleviate pod shatter include breeding shatter resistant strains, using mechanical techniques such as swathing during harvest and the use of adjuvants such as pod sealants during desiccation.

Thus, WO 99/15681 (Biogemma UK Limited) discloses a recombinant or isolated nucleic acid involved in the process of pod dehiscence. The nucleic acid is claimed to have application to all crops which lose seed pre-harvest due to cell wall separation events. Also disclosed is a method of regulating dehiscence, the method comprising the step of transforming or transfecting propagating material from a plant with the said nucleic acid. One disadvantage with this approach is finding the balance between shatter resistance whilst allowing the seed to be harvested. Another disadvantage is consumer resistance to genetically modified crops.

Swathing hastens drying of the crop and results in even ripening. Another method for drying the crop is application of chemical desiccants such as the herbicides glyphosate and diquat dibromide. The desiccant is sprayed onto the crop just before it reaches maturity to accelerate the ripening. Both methods can lead to immature and smaller seeds, as well as increased pod shatter if application of the method is timed incorrectly.

Pod sealants are designed to stop the pods from splitting open during ripening. Typical adjuvants are polymers that form a mesh around the pod protecting the pod during wind and rain conditions.

U.S. Pat. No. 4,447,984 (Sampson et al.) discloses a method for reducing or preventing premature release or scattering of seed due to splitting of seed-bearing bodies comprising applying to plants not more than 15 days before harvest a composition comprising di-1-p-menthene and/or polymeric di-1-p-menthene to provide a semi-permeable coating on said seed-bearing bodies. One disadvantage is that di-1-p-menthene is a naturally occurring material and cannot be sourced sustainably. Another disadvantage is that di-1-p-menthene is toxic to aquatic organisms, and causes skin irritation and dermatitis.

WO 2011/147721 (Lamberti SpA) discloses a method for preventing the premature opening of seed pods comprising the steps of: i) preparing an aqueous pod sealant composition comprising from 0.035 to 1.2 percent w/v of a carboxymethyl cellulose with a degree of polymerization of less than 1500, ii) spraying from 60 to 500 l/ha of said composition on pod-bearing crops within a month prior to harvesting. Unfortunately, the polymeric nature of the composition would be expected to lead to frequent blockage of the spray applicator nozzle on application.

WO 2016/046418 (Brandon products Limited) discloses a composition for use in reducing pod shattering, the composition comprising a salt of phosphorous acid. It is claimed that the composition can be applied to the growing plants before pods are formed as the effect is internal, rather than external. A disadvantage of this composition is that it accumulates in the crop during use.

Thus there still remains a need to provide a means to prevent pod shatter.

### SUMMARY OF THE INVENTION

The invention thus relates in a first aspect to use of cucurbituril, a derivative thereof, or a mixture thereof for reducing pod shattering in pod-bearing crops.

In a second aspect of the invention, a method for reducing pod shattering in pod-bearing crops is provided, the method comprising the application of cucurbituril, a derivative thereof, or a mixture thereof to a pod-bearing crop.

Rapeseed crop trials using a range of cucurbiturils as pod sealants have demonstrated comparative or improved crop yields compared to commercial products. The trials have also shown that the level of volunteers observed when using cucurbituril as a pod sealant is comparable to that of commercial products.

In a third aspect of the invention, a pod sealant composition comprising:

a) cucurbituril, a derivative thereof, or a mixture thereof; and

b) a desiccant.

### DETAILED DESCRIPTION OF THE INVENTION

The invention provides for use of cucurbituril, a derivative thereof, or a mixture thereof for reducing pod shattering in pod-bearing crops.

The invention further provides a method for reducing pod shattering in pod-bearing crops comprising the application of cucurbituril, a derivative thereof, or a mixture thereof to a pod-bearing crop.

Cucurbituril is a member of the cavitand family, and the general cucurbituril structure is based on the cyclic arrangement of glycoluril subunits linked by methylene bridges.

The preparation and purification of cucurbituril compounds is well described in the art. For example, Lagona et al. (Angew. Chem. Int. Ed., 2005, 44, 4844-4870) review the synthesis and properties of cucurbituril compounds, including derivatives, analogues and congeners within the cucurbituril family.

For example, cucurbit[8]uril (CB[8]; CAS 259886-51-6) is a barrel shaped container molecule which has eight repeat glycoluril units as shown in Structure 1 and an internal cavity volume of 479 cubic Angstroms. CB[8] is readily synthesised using standard techniques and is also available commercially (e.g. Sigma-Aldrich, MO, USA).

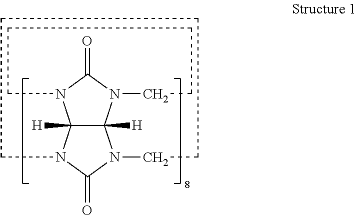

The cucurbituril may be selected from the group consisting of CB[5], CB[6], CB[7], CB[8], CB[10] and a mixture of two or more cucurbituril selected from the group consisting of CB[5], CB[6], CB[7], CB[8], CB[9], CB[10], CB[11], CB[12], CB[13] and CB[14]. Preferably the cucurbituril is CB[6].

Alternatively the cucurbituril is a mixture of two or more cucurbituril selected from the group consisting of CB[5], CB[6], CB[7], CB[8], CB[9], CB[10], CB[11], CB[12], CB[13] and CB[14]. Preferably the cucurbituril is CB[6].

When CB[5] is present in the mixture, the concentration of CB[5] may be from about 0.1 to about 99, more particularly from about 0.5 to about 75, more particularly from about 1 to about 50, more particularly from about 2 to about 40, more particularly from about 10 to about 30, more particularly from about 20 to about 25 percent by weight, based on the total weight of the cucurbituril in the mixture.

When CB[6] is present in the mixture, the concentration of CB[6] may be from about 0.1 to about 99, more particularly from about 1 to about 75, more particularly from about 5 to about 60, more particularly from about 40 to about 70, more particularly from about 35 to about 55 percent by weight, based on the total weight of cucurbituril in the composition.

When CB[7] is present in the mixture, the concentration of CB[7] may be from about 0.1 to 99, more particularly from about 5 to about 75, more particularly from about 10 to about 60, more particularly from about 20 to about 50, more particularly from about 20 to about 30 percent by weight, based on the total weight of cucurbituril in the composition. In one embodiment, the concentration of CB[7] is less than 35 percent by weight, based on the total weight of cucurbituril in the composition.

When CB[8] is present in the mixture, the concentration of CB[8] may be from about 0.1 to 99, more particularly from about 0.5 to about 75, more particularly from about 1 to about 30, more particularly about 2 to about 20, more particularly from about 3 to about 15 percent by weight, based on the total weight of cucurbituril in the composition.

The mixture may comprise at least two different cucurbiturils selected from CB[5], CB[6], CB[7] and CB[8]. Preferably when the mixture comprises at least two different cucurbiturils selected from CB[5], CB[6], CB[7] and CB[8], the total concentration of the CB[5], CB[6], CB[7] and/or CB[8] is greater than 75, more particularly greater than about 90, more particularly greater than about 99 percent by weight of the total weight of cucurbituril in the mixture. When the mixture comprises at least two different cucurbiturils selected from CB[5], CB[6], CB[7] and CB[8], the remaining components of the mixture may be selected from the group consisting of CB[4], CB[9] and CB[10] to CB[20], either as a single sized cucurbituril or as a mixture of these sizes.

The mixture may comprise CB[5], CB[6], CB[7] and CB[8], preferably 10-30 percent w/w total cucurbituril CB[5], 30-70 percent w/w total cucurbituril CB[6], 15-50 percent w/w total cucurbituril CB[7] and 3-30 percent w/w total cucurbituril CB[8]. More preferably the mixture comprises CB[5], CB[6], CB[7] and CB[8], more preferably 15-25 percent w/w total cucurbituril CB[5], 35-55 percent w/w total cucurbituril CB[6], 20-35 percent w/w total cucurbituril CB[7], 3-15 percent w/w total cucurbituril CB[8] and less than 1 percent by weight of cucurbiturils CB[9] and higher cucurbiturils.

The % weights of cucurbiturils described above are based on the total weight of cucurbituril (of all sizes) in the mixture.

A derivative of a cucurbituril is a structure having one, two, three, four or more substituted glycoluril units. A derivative of a cucurbituril compound is therefore represented by Structure 2,

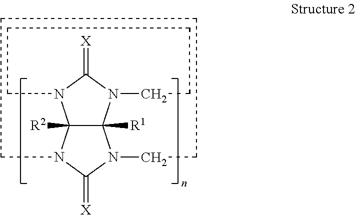

wherein n is an integer between 4 and 20; and for each glycoluril unit, each X is O, S or NR³; and —R¹ and —R² are each independently selected from —H and the following optionally substituted groups: —R³, —OH, —OR³, —COOH, —COOR³, —NH₂, —NHR³ and —N(R³)₂, where —R³ is independently selected from optionally substituted C₁₋₂₀ alkyl, C₆₋₂₀ aryl, and C₅₋₂₀ heteroaryl; or where —R¹ and/or —R² is —N(R³)₂, both —R³ together form a C₅₋₇ heterocyclic ring, or together —R¹ and —R² are C₄₋₆alkylene forming a C₆₋₈carbocyclic ring together with the uracil frame.

One of the glycoluril units may be a substituted glycoluril unit and thus —R¹ and —R² are each independently —H for n−1 of the glycoluril units.

n may be 5, 6, 7, 8, 9, 10, 11 or 12, preferably n is 5, 6, 7 or 8.

Each X may be O, alternatively each X is S.

R¹ and R² may be each independently H.

Optionally, for each unit one of R¹ and R² is H and the other is independently selected from —H and the following optionally substituted groups: —R³, —OH, —OR³, —COOH, —COOR³, —NH₂, —NHR³ and —N(R³)₂. Optionally, for one unit one of R¹ and R² is H and the other is independently selected from —H and the following optionally substituted groups: —R³, —OH, —OR³, —COOH, —COOR³, —NH₂, —NHR³ and —N(R³)₂. For this option, the remaining glycoluril units are such that R¹ and R² are each independently H.

—R³ may be C₁₋₂₀ alkyl, preferably C₁₋₆ alkyl. Preferred substituents on —R³ are selected from: —R⁴, —OH, —OR⁴, —SH, —SR⁴, —COOH, —COOR⁴, —NH₂, —NHR⁴ and —N(R⁴)₂, wherein —R⁴ is selected from optionally substituted C₁₋₂₀ alkyl, optionally substituted C₆₋₂₀ aryl, and optionally substituted C₅₋₂₀ heteroaryl. Preferably the substituents on —R³ are independently selected from —COOH and —COOR⁴.

Preferably, —R⁴ is not the same as —R³. Preferably, —R⁴ is unsubstituted.

Where —R¹ and/or —R² is —OR³, —NHR³ or —N(R³)₂, then —R³ is preferably C₁₋₆alkyl. Preferably, —R³ is substituted with a substituent —OR⁴, —NHR⁴ or —N(R⁴)₂. Each —R⁴ is C₁₋₆alkyl and is itself preferably substituted.

Cucurbiturils are often derivatised to improve their suspendability in a carrier, and more generally their formulation and handling.

In practice, cucurbiturils and derivatives thereof are often present with ionic species which are manufacturing residues and/or additives. Additives may be used to improve the solubility of cucurbituril in water, for example, ammonium sulphate, ammonium hydroxide, ammonium carbonate or ammonium nitrate, particularly ammonium sulphate. Preferably the molar ratio of additive to improve water solubility to cucurbituril is 0.2:1 to 20:1, preferably 1:1 to 10:1, most preferably 1:1 to 5:1.

Thus the ionic species may be selected from the group consisting of a halide, a sulphate, a formate, a carbonate, a nitrate, an acetate, a phosphate, a hydroxide, alkali metal ions, alkaline earth metal ions, and quaternary ammonium salts. The corresponding salt may be selected from the group consisting of sodium carbonate, ammonium carbonate, sodium hydrogen carbonate, ammonium chloride, lithium chloride, sodium chloride, potassium chloride, calcium chloride, ammonium bromide, lithium bromide, sodium bromide, potassium bromide, calcium bromide, potassium hydroxide, sodium hydroxide, lithium hydroxide, ammonium hydroxide, rubidium hydroxide, caesium hydroxide, calcium hydroxide, magnesium hydroxide, strontium hydroxide, barium hydroxide, lithium iodide, sodium iodide, potassium iodide, calcium iodide, ammonium nitrate, sodium sulphate, potassium sulphate, lithium sulphate, calcium sulphate, ammonium sulphate ammonium methane sulphonate, lithium methane sulphonate, sodium methane sulphonate, potassium methane sulphonate, and calcium methane sulfonate.

The application concentration of the cucurbituril, a derivative thereof, or a mixture thereof is 0.025-100 g/hectare, preferably 0.5-2.5 g/hectare, most preferably 0.5-1.5 g/hectare. Application is typically by spraying the pod-bearing crop.

The cucurbituril, a derivative thereof, or a mixture thereof is preferably applied to a crop simultaneously with a desiccant, such as a glyphosate, glufosinate or diquat. The diquat is preferably the dibromide derivative.

The pod-bearing crop is selected from those in which it is desired to inhibit the impact of dehiscence. Thus the pod-bearing crop may be selected from the plant family Fabaceae, Linacaea or Brassicaceae. More particularly, the pod-bearing crop may be selected from the group consisting of Vicia, Phaseolus, Vigna, Cicer, Pisum, Lathyrus, Lens, Lablab, Glycine, Psophocarpus, Cajanus, Mucuna, Cyamopsis, Canavalia, Macrotyloma, Lupinus, Linum and Brassica. Even more particularly, the pod-bearing crop may be selected from the group consisting of Vicia faba (fava bean), Phaseolus coccineus (runner bean), Phaseolus lunatus (lima bean), Phaseolus vulgaris (common bean), Vigna angularis (adzuki bean), Vigna Radiata (mung bean), Vigna unguiculata (cowpea), Cicer arietinum (chickpea), Pisum sativum (pea), Lens culinaris (lentil), Glycine max (soybean), Cajanus cajan (pigeon pea), Cyamopsis tetragonoloba (guar), Lupinus albus (lupine bean), Linum usitatissimum (linseed) and Brassica napus (rapeseed). Preferably the pod-bearing crop is Brassica napus (rapeseed) or Glycine max (soybean).

For the purposes of this specification, the term Brassica napus (rapeseed) includes the closely related Brassica rapa and canola, a commercially important cultivar of rapeseed bred with low erucic acid levels.

The cucurbituril, a derivative thereof, or a mixture thereof is typically applied to a pod-bearing crop when weight average seed moisture is less than 40, preferably 35, most preferably 30 percent w/w. In addition and alternatively, the cucurbituril, a derivative thereof, or a mixture thereof may be applied to a pod-bearing crop when at least 20, preferably at least 25, more preferably at least 30 percent of the pods are ripe. At this point in the growth cycle, the majority of seeds have reached their physiological maturity.

The invention also provides a pod sealant composition comprising:

a) cucurbituril, a derivative thereof, or a mixture thereof; and

b) a desiccant.

The description set forth hereinabove relating to cucurbituril, a derivative thereof, applies mutatis mutandis to the pod sealant composition.

The composition may comprise 0.01-100 g/l, more preferably 0.01-10 g/l cucurbituril, a derivative thereof, or a mixture thereof.

The desiccant may be selected from glyphosate, glufosinate or diquat. The diquat is preferably the dibromide derivative.

The composition may further comprise any one of a herbicide, a fungicide, an insecticide, a fertilizer, micronutrients, an emulsifier, a wetting agent, a thickener, a preservative, a suspending agent, an anti-drift agent, ionic species, or a mixture thereof. In addition, the composition typically further comprises a carrier. Examples of carriers are water, emulsions including multiple emulsions, and organic solvents. The carrier may be a solid particulate material.

Where the carrier comprises water, the composition may have a pH of 2-8, preferably 3-6.

The invention is now illustrated by way of an example.

### EXAMPLE 1

A mixture of cucurbiturils was prepared by preparing a solution of glycoluril (111 g, 0.781 moles) in water (123 ml, 6.833 moles) under stirring. Sulphuric acid (125 mL, 2.34 moles) and formalin (116 mL, 1.56 moles) were added to the solution of glycoluril and the mixture heated to 100° C. and held under stirring for 14 hours. The reaction mixture was then cooled to room temperature over a 3 hour time period and the cooled reaction mixture precipitated into methanol (1000 ml). The precipitate was filtered, slurried with methanol (1000 ml), filtered again and dried.

The mixture was determined using nuclear magnetic resonance (¹H NMR) imaging to be 20-25 percent by weight CB[5], 45-55 percent by weight CB[6], 20-25 percent by weight of CB[7], 5-8 percent by weight of CB[8], and less than 1 percent by weight CB[9] and higher cucurbiturils, based on the total weight of cucurbituril in the mixture.

Cucurbit[6]uril, cucurbit[7]uril, cucurbit[8]uril and the mixture of cucurbiturils without cucurbit[8]uril were each isolated from the mixture of cucurbiturils in accordance with the method set forth in Kim et al. (J. Am. Chem. Soc., 2000, 122 (3), 540-541). Briefly, the method relies on the different solubility of different cucurbiturils in various solvents.

Saturated aqueous solutions (pH 5, NaOH) of cucurbit[6]uril, cucurbit[7]uril, cucurbit[8]uril, of the mixture of cucurbiturils and the mixture of cucurbiturils without cucurbit[8]uril were prepared and then tested against commercial polymeric pod sealants containing carboxylated synthetic latex polymer (P1) and different preparations of carboxylated styrene butadiene co-polymer (P2 and P3) on a rapeseed (DK Extrovert (Monsanto Company)) crop in Sherburn, North Yorkshire, UK.

The seed was sown 18 Aug. 2015 at a rate of 3.5 kg/ha which equates to a density of 60 seeds per square metre. On 18 Jul. 2016, four 12 m×2 m plots were randomly chosen and treated by spraying with the solutions described in Table 1. At this time, the crop growth stage was 82-84 (BBCH decimal system growth stage scale), i.e., about 30 percent of the pods were ripe and the weight average seed moisture was about 30-35 percent w/w (ISO 665:2000 Oilseeds: Determination of moisture and volatile matter content)). The crop height was 130-150 cm. The level of treatment was kept about the same for all the treatments. The desiccant glyphosate was used in all but one of the cases in order to even up the ripening of the pods on the main and lateral racemes. Table 1 summarises the various test pod sealing treatments.

TABLE 1
Pod sealing treatments (“3 + 1” means 3 l/hectare of the first treatment
component and 1 l/hectare of the second treatment component)

| | | Treatment |
|---|---|---|
| Treatment | | application rate |
| number | Treatment description | (l/hectare) |
| 1 | Untreated | n/a |
| 2 | Glyphosate | 3 |
| 3 | Glyphosate + P1 | 3 + 1 |
| 4 | Glyphosate + P2 | 3 + 1 |
| 5 | Glyphosate + P3 | 3 + 1 |
| 6 | Glyphosate + cucurbit[6]uril | 3 + 1 |
| 7 | Glyphosate + cucurbit[7]uril | 3 + 1 |
| 8 | Glyphosate + cucurbit[8]uril | 3 + 1 |
| 9 | Glyphosate + the mixture of cucurbiturils | 3 + 1 |
| 10 | Glyphosate + the mixture of cucurbiturils | 3 + 1 |
| | without cucurbit[8]uril | |

The crop was harvested by the direct heading method on 13 Aug. 2016. The yield (metric ton/hectare) of rapeseed and the number of volunteer seeds per square metre (manually counted) were determined and the results are set forth in Table 2.

TABLE 2
The yield (metric ton/hectare) and the number of volunteer
seeds per square metre (manually counted) for the pod sealing
treatments set forth in Table 1 with statistical analysis

| Treatment | Yield (metric | Yield (% | Volunteer yield |
|---|---|---|---|
| number | ton/hectare) | treatment 2) | (seeds/square metre) |
| 1 | 2.749 a | 103.51 a | 223.8 ab |
| 2 | 2.656 a | 100.00 a | 235.0 a |
| 3 | 2.709 a | 102.20 a | 181.3 c |
| 4 | 2.804 a | 105.61 a | 182.5 c |
| 5 | 2.843 a | 107.08 a | 198.8 bc |
| 6 | 2.794 a | 105.38 a | 207.5 abc |
| 7 | 2.795 a | 105.30 a | 202.5 bc |
| 8 | 2.823 a | 106.38 a | 226.3 ab |
| 9 | 2.950 a | 111.14 a | 200.0 bc |
| 10 | 2.741 a | 103.34 a | 203.8 bc |

Whilst statistically there is no difference in yield between any of the treatments, the trend is that treatment 9 out performs any of the other treatments, and that all the treatments including curcubiturils perform at least as well as the commercial pod sealants.

### EXAMPLE 2

A saturated aqueous solution (pH 5) of a mixture of cucurbiturils was prepared by adding 0.45 g cucurbit[n]uril powder to 500 mL of deionised water, which was then stirred with an overhead mixer to produce an opaque white suspension containing undissolved cucurbituril which settled upon standing.

A saturated aqueous solution (pH 5) containing a higher amount of dissolved cucurbituril was prepared by adding 0.12 g ammonium sulphate (Acros Organics) and 0.60 g cucurbit[n]uril powder to 500 mL deionised water whilst stirring with an overhead mixer to produce an opaque white suspension containing a small amount of undissolved cucurbituril which settled upon standing.

Thus ammonium sulphate was observed to increase the solubility of cucurbituril in water.

1. (canceled)

2. A method for reducing pod shattering in pod-bearing crops comprising the application of cucurbituril, a derivative thereof, or a mixture thereof to a pod-bearing crop.

3. The method according to claim 2, wherein the cucurbituril is selected from the group consisting of CB[5], CB[6], CB[7], CB[8], CB[10] and a mixture of two or more cucurbituril selected from the group consisting of CB[5], CB[6], CB[7], CB[8], CB[9], CB[10], CB[11], CB[12], CB[13] and CB[14].

4. The method according to claim 3, wherein the cucurbituril is CB [6].

5. The method according to claim 3, wherein the cucurbituril is the mixture of two or more cucurbituril selected from the group consisting of CB[5], CB[6], CB[7], CB[8], CB[9], CB[10], CB[11], CB[12], CB[13] and CB[14].

6. The method according to claim 2, wherein the application concentration of the cucurbituril, a derivative thereof, or a mixture thereof is 0.025-100 g/hectare.

7. The method according to claim 2, wherein the pod-bearing crop is selected from the plant family Fabaceae, Linacaea or Brassicaceae.

8. The method according to claim 7, wherein the pod-bearing crop is selected from the group consisting of Vicia, Phaseolus, Vigna, Cicer, Pisum, Lathyrus, Lens, Lablab, Glycine, Psophocarpus, Cajanus, Mucuna, Cyamopsis, Canavalia, Macrotyloma, Lupinus, Linum and Brassica.

9. The method according to claim 8, wherein the pod-bearing crop is selected from the group consisting of Vicia faba (fava bean), Phaseolus coccineus (runner bean), Phaseolus lunatus (lima bean), Phaseolus vulgaris (common bean), Vigna angularis (adzuki bean), Vigna Radiata (mung bean), Vigna unguiculata (cowpea), Cicer arietinum (chickpea), Pisum sativum (pea), Lens culinaris (lentil), Glycine max (soybean), Cajanus cajan (pigeon pea), Cyamopsis tetragonoloba (guar), Lupinus albus (lupine bean), Linum usitatissimum (linseed) and Brassica napus (rapeseed).

10. The method according to claim 9, wherein the pod-bearing crop is Brassica napus (rapeseed) or Glycine max (soybean).

11. The method according to claim 2, wherein the cucurbituril, a derivative thereof, or a mixture thereof is applied to a pod-bearing crop when weight average seed moisture is less than 40 percent w/w.

12. A pod sealant composition comprising:
a) cucurbituril, a derivative thereof, or a mixture thereof; and
b) a desiccant.

13. The composition according to claim 12, wherein the cucurbituril is selected from the group consisting of CB[5], CB[6], CB[7], CB[8], CB[10] and a mixture of two or more cucurbituril selected from the group consisting of CB[5], CB[6], CB[7], CB[8], CB[9], CB[10], CB[11], CB[12], CB[13] and CB[14].

14. The composition according to claim 13, wherein the cucurbituril is CB[6].

15. The composition according to claim 13, wherein the cucurbituril is the mixture of two or more cucurbituril selected from the group consisting of CB[5], CB[6], CB[7], CB[8], CB[9], CB[10], CB[11], CB[12], CB[13] and CB[14].

16. The composition according to claim 12, wherein the composition comprises 0.01-100 g/l cucurbituril, a derivative thereof, or a mixture thereof.

17. The composition according to claim 12, wherein the desiccant is selected from glyphosate, glufosinate or diquat.

18. The composition according to claim 12, wherein the composition further comprises any one of a herbicide, a fungicide, an insecticide, a fertilizer, micronutrients, an emulsifier, a wetting agent, a thickener, a preservative, a suspending agent, an anti-drift agent, ionic species or a mixture thereof.

19. The composition according to claim 12, wherein the composition further comprises an additive for increasing the solubility of cucurbituril in water.

20. The composition according to claim 12, wherein the composition further comprises a carrier.

21. The composition according to claim 12, wherein the composition has a pH of 2-8.
